(19) United States
(12) Patent Application Publication
(10) Pub. No.: US 2025/0092461 A1
(43) Pub. Date: Mar. 20, 2025

(54) PERSONALIZED CANCER MANAGEMENT AND MONITORING BASED ON DNA METHYLATION CHANGES IN CELL-FREE DNA

(71) Applicant: NUCLEIX LTD., Rehovot (IL)

(72) Inventors: Danny FRUMKIN, Rehovot (IL); Adam WASSERSTROM, Ness Ziona (IL)

(73) Assignee: NUCLEIX LTD., Rehovot (IL)

(21) Appl. No.: 18/727,943

(22) PCT Filed: Jan. 12, 2023
(86) PCT No.: PCT/IL2023/050040

§ 371 Date: Jul. 10, 2024

Related U.S. Application Data
(60) Provisional application No. 63/299,023, filed on Jan. 13, 2022.

(51) Int. Cl. C12Q 1/6886; C12Q 1/6851

(52) U.S. Cl. CPC C12Q 1/6886; C12Q 1/6851; C12Q 2600/118; C12Q 2600/154

(57) ABSTRACT

Methods and systems for personalized cancer management and monitoring are provided, such as evaluating minimal residual disease (MRD), monitoring tumor recurrence, predicting and monitoring response to treatment and prognosis, based on detection and tracking of tumor-associated DNA methylation changes in cell-free DNA samples, particularly cell-free DNA from plasma samples.

### FIELD OF THE INVENTION

The present invention relates to methods and systems for personalized cancer management and monitoring, such as evaluating minimal residual disease (MRD), monitoring tumor recurrence, predicting and monitoring response to treatment and prognosis, based on detection and tracking of tumor-associated DNA methylation changes in cell-free DNA samples, particularly cell-free DNA from plasma samples.

### BACKGROUND OF THE INVENTION

Tumors are known to release DNA fragments, or “cell-free DNA”, into body fluids and consequently genetic and epigenetic changes of tumor derived DNA molecules can be detected in “liquid biopsies” obtained from body fluids such as blood plasma and urine. In contrast to traditional biopsies, liquid biopsies are non-invasive and may better represent the full genetic spectrum of tumor sub-clones. Detection of genetic and epigenetic changes associated with cancer in liquid biopsies holds great promise for early detection, prognosis, and therapeutic surveillance. For example, detecting the presence of tumor-derived cfDNA in body fluid samples could indicate that there are cancer cells present in the subject's body after treatment, and thus allow monitoring recurrence, response to treatment and more.

However, in order to detect tumor derived DNA in liquid biopsies, ultra-sensitive biochemical methods are required, because the tumor DNA can be present in extremely low quantities in relation to the large background of normal DNA. In addition, characteristics of tumor-derived DNA can vary greatly between different types of cancer and even between different patients of the same cancer type, and thus designing an assay for tracking tumor-derived DNA that is suitable for a broad range of cancer types and variations is challenging.

Assays for personalized monitoring tumor recurrence, metastasis development, response to treatment and more based on detecting and tracking of tumor-derived circulating DNA are sought.

Bratman et al. (2020) Nature Cancer, 1: 873-881, report an amplicon-based bespoke circulating tumor DNA (ctDNA) detection platform for prognostication and response monitoring in patients treated with the anti-PD-1 monoclonal antibody, pembrolizumab. Whole-exome sequencing (WES) was performed on protocol-mandated fresh-frozen tumor biopsy tissue obtained within 4 weeks of treatment initiation or, if insufficient material was available from the protocol-mandated biopsy, on archival tumor tissue obtained 1-83 months before treatment initiation. In each case, matched germline WES was performed to identify tumor-specific somatic mutations. For each patient, up to 16 clonal somatic mutations were selected for personalized ctDNA assay design.

US 2016/0032396 and EP 3421613 B1 disclose methods for creating a selector of mutated genomic regions and for using the selector set to analyze genetic alterations in a cell-free nucleic acid sample. The methods can be used to measure tumor-derived nucleic acids in a blood sample from a subject and thus to monitor the progression of disease in the subject. The methods can also be used for cancer screening, cancer diagnosis, cancer prognosis, and cancer therapy designation.

U.S. Pat. No. 10,450,611 discloses methods and systems for personalized genetic testing of a subject. In some embodiments, a sequencing assay is performed on a biological sample from the subject, which then leads to genetic information related to the subject. Next, nucleic acid molecules are array-synthesized or selected based on the genetic information derived from data of the sequencing assay. At least some of the nucleic acid molecules may then be used in an assay which may provide additional information on one or more biological samples from the subject or a biological relative of the subject.

US 2019/0316184 discloses, inter alia, a method for monitoring and detection of early relapse or metastasis of breast cancer, bladder cancer, or colorectal cancer, comprising generating a set of amplicons by performing a multiplex amplification reaction on nucleic acids isolated from a sample of blood or urine or a fraction thereof from a patient who has been treated for a breast cancer, bladder cancer, or colorectal cancer, wherein each amplicon of the set of amplicons spans at least one single nucleotide variant locus of a set of patient-specific single nucleotide variant loci associated with the breast cancer, bladder cancer, or colorectal cancer; and determining the sequence of at least a segment of each amplicon of the set of amplicons that comprises a patient-specific single nucleotide variant locus, wherein detection of one or more patient-specific single nucleotide variants is indicative of early relapse or metastasis of breast cancer, bladder cancer, or colorectal cancer.

US 2020/0248266 discloses subject-specific methods for detecting recurrence of tumors based on an understanding of the clonal/subclonal mutation profile of the subject's tumor and detection of the mutations in their cell-free DNA (cfDNA), typically by multiplex PCR of tumor mutations such as single nucleotide variants (SNVs).

Hitherto described methods for personalized cancer monitoring based on detection and tracking of tumor-derived circulating DNA have a number of drawbacks, where some require whole genome analysis of tumor samples and re-design of a panel of markers for each and every patient individually, some involve costly sequencing and some are applicable only to a limited group of cancer types.

There remains a need for improved personalized cancer assays, which provide robust performance, are cost-effective, simple to operate and suitable for a broad range of cancer types.

### SUMMARY OF THE INVENTION

The present invention provides methods and systems for establishing personalized panels of cancer-associated DNA methylation markers, for tracking and measuring tumor burden in cancer patients non-invasively. The methods and systems of the present invention are based on testing the methylation of a pre-defined set of cancer-associated marker loci in DNA samples comprising tumor DNA of a subject diagnosed with cancer, and based on this measurement selecting from the set a personalized subset of marker loci which are informative for the subject's cancer. The cancer burden can then be monitored non-invasively by measuring methylation of the selected subset of marker loci in cell-free DNA samples of the subject.

In some embodiments, the methods of the present invention are tumor-informed, and carried out using methylation information derived from a tumor sample of the subject. According to these embodiments, the methylation values of a defined set of cancer-associated marker loci is analyzed in tumor DNA of a subject diagnosed with cancer. Next, a subset of marker loci that show significant difference in methylation values between the tumor DNA and normal non-cancer DNA is selected from the broader set. This subset of represents informative marker loci for the subject's tumor out of the complete set. The tumor burden is assessed or monitored non-invasively by testing the selected subset of genomic loci in cell-free DNA (e.g. from plasma sample(s)) of the subject.

In other embodiments, the methods of the present invention are tumor-uninformed, and based on methylation information derived from cell-free DNA from a plasma sample of the subject. The tumor-uninformed configuration does not require analyzing a tumor sample in order to select a subset of informative markers. The assay is based on analysis of cell-free DNA and DNA from peripheral blood leukocytes (PBLs) of a subject diagnosed with cancer. In some embodiments, the methylation values of the defined set of cancer-associated marker loci are measured in cell-free DNA from a plasma sample and in DNA from PBLs of a subject diagnosed with cancer. Next, a subset of informative marker loci is selected from the broad set of marker loci. The tumor burden is monitored non-invasively by testing the selected subset of genomic loci in cell-free DNA sample(s) of the subject (e.g., plasma DNA).

The present invention therefore provides a semi-custom assay for measuring tumor burden non-invasively which are advantageous over existing methods. For example, the methods and systems of the present invention do not require custom design of a set of markers for each subject individually, but rather just selecting a subset of markers which are the most informative for each subject out of a pre-defined set of markers, thus saving time and costs. In addition, measuring methylation values of the selected subset of markers in cell-free DNA samples can be carried out by real-time PCR using primers and probes that were previously validated, thus providing robust performance of the assay. As a further advantage, the methods and system do not require high-throughput sequencing for either subset selection or clinical assay, thus saving time and costs. The tumor-uninformed configuration of the assay does not require to analyze a tumor sample, and is therefore more widely applicable and saves time and costs.

The pre-defined set of marker loci according to the present invention typically comprises at least 10 marker loci, for example, at least 11, 12, 13, 14, 15, 16, 16, 20 marker loci. Each possibility represents a separate embodiment of the present invention. In some embodiments, the pre-defined set of marker loci according to the present invention comprises at least 30 marker loci, at least 40 marker loci or at least 50 marker loci. Each possibility represents a separate embodiment of the present invention. In some embodiments, the pre-defined set of marker loci according to the present invention comprises between 10-200 marker loci, for example between 10-100 marker loci, between 15-100 marker loci between 20-100 marker loci, between 30-100 marker loci, between 40-100 marker loci or between 50-100 marker loci. Each possibility represents a separate embodiment of the present invention.

A selected subset of marker loci according to the present invention may include one or a plurality of markers. The subset includes a smaller number of marker loci compared to the complete broad set, which show differential methylation between tumor ad normal DNA. In some embodiments, the most informative loci are selected, for example, loci that show the highest hypermethylation in the tumor DNA compared to normal DNA. As a further example, loci which show the highest signal to noise ratio are selected, namely, loci which show the highest ratio between methylation values in the tumor DNA and methylation values in normal DNA. In some embodiments, the subset of marker loci comprises 1-20 marker loci, 1-10 marker loci, 5-20 marker loci or 10-20 marker loci. Each possibility represents a separate embodiment of the present invention.

The methods of the present invention may be carried out in parallel or in combination with measuring additional analytes, such as mutations.

According to one aspect, the present invention provides a method for establishing a personalized panel of cancer-associated DNA methylation markers, the method comprising:
(a) determining in a tumor DNA sample of a subject diagnosed with cancer a methylation value for each marker locus in a pre-defined set of marker loci comprising pan-cancer marker loci, cancer-specific marker loci which are specific to the cancer of the subject, or a combination thereof;
(b) comparing the methylation values determined in step (a) to corresponding methylation values of the marker loci in normal non-cancer DNA; and
(c) selecting a subset of marker loci out of the set of marker loci that show a significant difference in methylation values in the tumor DNA compared to the corresponding normal non-cancer DNA, thereby establishing a personalized panel of cancer-associated DNA methylation markers.

In some embodiments, the subject is diagnosed with a solid tumor cancer, and wherein the methylation values of the marker loci in normal non-cancer DNA are methylation values determined in DNA extracted from peripheral blood leukocytes of the subject.

In some embodiments, the subject is diagnosed with a hematological cancer, and wherein the methylation values of the marker loci in normal non-cancer DNA are methylation values determined in DNA extracted from urine or saliva samples of the subject.

In some embodiments, the subject is diagnosed with a hematological cancer, and wherein the methylation values of the marker loci in normal non-cancer DNA are reference methylation values determined based on a plurality of DNA samples of healthy subjects without a hematological cancer.

In some embodiments, methylation values are determined using methylation-sensitive enzymatic digestion of DNA followed by high-throughput sequencing.

In some embodiments, methylation values are determined using methylation-sensitive enzymatic digestion of DNA followed by quantitative PCR and analysis of amplification products.

In some embodiments, a method for guiding therapy of a subject diagnosed with cancer is provided, the method comprising:
(i) establishing for the subject a personalized panel of cancer-associated DNA methylation markers as disclosed herein; and
(ii) analyzing the methylation values of the selected subset of marker loci in one or more cell-free DNA samples of the subject to guide therapy of the subject, wherein methylation values of the selected subset of marker loci in the cell-free DNA sample are indicative of tumor burden in the subject.

In some embodiments, the cell-free DNA sample is cell-free DNA extracted from a plasma sample.

In some embodiments, guiding therapy of the subject comprises providing an indication of recurrence of the cancer, and wherein analyzing the methylation values of the selected subset of marker loci in one or more cell-free DNA samples of the subject comprises:
(i) determining baseline methylation values of the selected subset of marker loci in a first cell-free DNA sample of the subject collected at a first time point after administration of a treatment;
(ii) determining methylation values of the selected subset of marker loci in a second cell-free DNA sample of the subject collected at a second later time point after administration of the treatment; and
(iii) comparing the methylation values determined in the second cell-free DNA sample to the baseline methylation values in order to provide an indication whether the cancer recurred, wherein an increase in methylation values is indicative of cancer recurrence;
(iv) optionally repeating steps (ii)-(iii) for cell-free DNA samples collected at additional time points after administration of the treatment, and further comparing the methylation values determined at a late time point to methylation values determined at earlier time points, wherein an increase in methylation values is indicative of cancer recurrence.

In some embodiments, the method further comprising:
administering to the subject active cancer surveillance and follow-up testing when the indication for cancer recurrence is positive, for definitive diagnosis of cancer recurrence and optionally monitoring the progression of the cancer, wherein the cancer surveillance and follow-up testing comprises one or more of blood tests, urine tests, cytology, imaging, endoscopy and biopsy.

In some embodiments, the method further comprising administering treatment when the definitive diagnosis shows cancer recurrence, wherein the treatment comprises one or more of surgical resection, chemotherapy, radiation therapy, immunotherapy, and targeted therapy

In some embodiments, guiding therapy of the subject comprises assessing response to treatment, and wherein analyzing the methylation values of the selected subset of marker loci in one or more cell-free DNA samples of the subject comprises:
(i) determining methylation values of the selected subset of marker loci in a first cell-free DNA sample of the subject collected before administration of a treatment;
(ii) determining methylation values of the selected subset of marker loci in a second cell-free DNA sample of the subject collected after administration of the treatment; and
(iii) comparing the methylation values determined after treatment to the methylation values determined before treatment in order to assess response to the treatment, wherein a decrease in methylation values is indicative of a positive response to the treatment; and
(iv) optionally repeating steps (ii)-(iii) for cell-free DNA samples collected at additional time points after administration of the treatment, and further comparing the methylation values determined at a late time point to methylation values determined at earlier time points in order to monitor response to the treatment, wherein an increase in methylation values is indicative of a positive response to the treatment.

In some embodiments, the treatment comprises surgery and the second cell-free DNA sample is a sample taken after the surgery.

In some embodiments, the treatment is a treatment administered in cycles and the second cell-free DNA sample is one or more samples taken after one or more cycles.

In some embodiments, the treatment is a treatment administered in cycles and the second cell-free DNA sample is a sample taken after the cycles are completed.

In some embodiments, guiding therapy of the subject comprises predicting response to a treatment, and wherein analyzing methylation values of the selected subset of marker loci in one or more cell-free DNA samples of the subject comprises:
(i) determining methylation values of the selected subset of marker loci in a cell-free DNA sample of the subject collected before administration of the treatment; and
(ii) comparing the methylation values determined in the cell-free DNA sample of the subject to reference methylation values determined in a plurality of reference subjects that received the treatment, to classify the likelihood of the subject to positively respond to the treatment.

In some embodiments, a method of administering a cancer treatment to a subject diagnosed with cancer is provided, the method comprising:
classifying the likelihood of the subject to respond positively to the treatment as disclosed herein; and
administering the treatment to the subject when the subject is classified as likely to positively respond to the treatment.

In some embodiments, guiding therapy of the subject comprises classifying minimal residual disease after administration of a treatment, and wherein analyzing methylation values of the selected subset of marker loci in one or more cell-free DNA samples of the subject comprises:
(i) determining methylation values of the selected subset of marker loci in a first cell-free DNA sample of the subject collected before administration of the treatment;
(ii) determining methylation values of the selected subset of marker loci in a second cell-free DNA sample of the subject collected after administration of the treatment; and
(iii) comparing the methylation values determined after treatment to the methylation values determined before treatment and to methylation values in a normal non-cancer DNA, to classify minimal residual disease.

In some embodiments, the treatment comprises surgery and the second cell-free DNA sample is a sample taken after the surgery.

In some embodiments, the treatment is a treatment administered in cycles and the second cell-free DNA sample is a sample taken after the cycles are completed.

In some embodiments, establishing the personalized panel of cancer-associated DNA methylation markers is carried out using methylation-sensitive enzymatic digestion of DNA followed by high-throughput sequencing, and analyzing the methylation values of the selected subset of marker loci in one or more cell-free DNA samples is carried out using methylation-sensitive enzymatic digestion of DNA followed by quantitative PCR and analysis of amplification products.

In some embodiments, establishing the personalized panel of cancer-associated DNA methylation markers and analyzing the methylation values of the selected subset of marker loci in one or more cell-free DNA samples are carried out using methylation-sensitive enzymatic digestion of DNA followed by quantitative PCR and analysis of amplification products.

In some embodiments, determining a methylation value for a marker locus using methylation-sensitive enzymatic digestion of DNA followed by quantitative PCR and analysis of amplification products comprises:
subjecting the DNA to digestion with at least one methylation-sensitive restriction endonuclease recognizing a sequence within the marker locus that is differentially methylated between cancer and non-cancer DNA, thereby obtaining restriction endonuclease-treated DNA;
co-amplifying from the restriction endonuclease-treated DNA the marker locus and a control locus, thereby generating an amplification product for each locus;
determining a signal intensity for each generated amplification product; and
calculating a ratio between the signal intensities of the amplification products of the marker locus and the control locus, thereby determining a methylation value for the marker locus.

In some embodiments, the step of subjecting the DNA to digestion with at least one methylation-sensitive restriction endonuclease is performed using a plurality of methylation-sensitive restriction endonucleases.

In some embodiments, the control locus is a locus devoid of a recognition sequence of the methylation-sensitive restriction endonuclease.

In some embodiments, the step of co-amplifying from the restriction endonuclease-treated DNA the marker locus and a control locus is performed using real-time PCR.

In some embodiments, the step of co-amplifying from the restriction endonuclease-treated DNA the marker locus and a control locus comprises adding fluorescent probes for assisting in detecting the amplification products of the marker locus and the control locus.

In some embodiments, the ratio between the signal intensities of the amplification products of the marker locus and the control locus is calculated by determining the quantification cycle (Cq) for each locus and calculating 2(Cq control locus−Cq marker locus).

In some embodiments, the method further comprising preparing a report in paper or electronic form based on the methylation values, and optionally communicating the report to the subject and/or a healthcare provider of the subject.

In some embodiments, a method for personalized analysis of cancer-related methylation changes in DNA samples of a subject diagnosed with cancer is provided, the method comprising:
(A) providing a set of reagents for quantifying methylation of a pre-defined set of marker loci in DNA samples, the pre-defined set of marker loci comprises pan-cancer marker loci, cancer-specific marker loci which are specific to the cancer of the subject, or a combination thereof;
(B) determining in a tumor DNA sample of the subject a methylation value for each marker locus in the pre-defined set, and identifying a subset of marker loci from the set that show a significant difference in methylation values in the tumor DNA compared to corresponding methylation values of the marker loci in normal non-cancer DNA;
(C) selecting from the set of reagents provided in step (A) those reagents that are suitable for quantifying methylation of the subset of marker loci identified in step (B); and
(D) using the selected reagents for analyzing the methylation values of the subset of marker loci in one or more cell-free DNA samples of the subject, thereby performing a personalized analysis of cancer-related methylation changes in DNA samples of the subject.

In some embodiments, the method comprising:
(A) providing a set of primers and probes for real-time PCR amplification and detection of the pre-defined set of marker loci in DNA samples, the pre-defined set of marker loci comprises pan-cancer marker loci, cancer-specific marker loci which are specific to the cancer of the subject, or a combination thereof, wherein each marker locus contains at least one restriction site of at least one methylation-sensitive restriction endonuclease that is differentially methylated between cancer and non-cancer DNA;
(B) determining in a tumor DNA sample of the subject a methylation value for each marker locus in the pre-defined set, and identifying a subset of marker loci from the set that show a significant difference in methylation values in the tumor DNA compared to corresponding methylation values of the marker loci in normal non-cancer DNA;
(C) selecting from the set of primers and probes provided in step (A) those primers and probes that are suitable for amplification and detection of the subset of marker loci identified in step (B); and
(D) using the selected primers and probes, in combination with the at least one methylation-sensitive restriction endonuclease, for analyzing the methylation values of the subset of marker loci in one or more cell-free DNA samples of the subject, thereby performing a personalized analysis of cancer-related methylation changes in DNA samples of the subject.

In some embodiments, a method for establishing a personalized panel of cancer-associated DNA methylation markers is provided, the method comprising:
(a) determining in cell-free DNA extracted from a plasma sample of a subject diagnosed with cancer a methylation value for each marker locus in a pre-defined set of marker loci comprising pan-cancer marker loci, cancer-specific marker loci which are specific to the cancer of the subject, or a combination thereof;
(b) comparing the methylation values determined in step (a) to corresponding methylation values of the marker loci in normal non-cancer DNA; and
(c) selecting a subset of marker loci out of the set of marker loci that show a significant difference in methylation values in the cell-free DNA compared to the corresponding normal non-cancer DNA, thereby establishing a personalized panel of cancer-associated DNA methylation markers.

In some embodiments, the subject is diagnosed with a solid tumor cancer, and wherein the methylation values of the marker loci in normal non-cancer DNA are methylation values determined in DNA extracted from peripheral blood leukocytes of the subject.

In some embodiments, the subject is diagnosed with a hematological cancer, and wherein the methylation values of the marker loci in normal non-cancer DNA are methylation values determined in DNA extracted from urine or saliva samples of the subject.

In some embodiments, the subject is diagnosed with a hematological cancer, and wherein the methylation values of the marker loci in normal non-cancer DNA are reference methylation values determined based on a plurality of DNA samples of healthy subjects without a hematological cancer.

In some embodiments, a method for guiding therapy of a subject diagnosed with cancer is provided, the method comprising:
(i) establishing for the subject a personalized panel of cancer-associated DNA methylation markers according to any one of claims 32-35; and
(ii) analyzing the methylation values of the selected subset of marker loci in one or more cell-free DNA samples of the subject to guide therapy of the subject, wherein methylation values of the selected subset of marker loci in the cell-free DNA sample are indicative of tumor burden in the subject.

These and further aspects and features of the present invention will become apparent from the detailed description, examples and claims which follow.

### BRIEF DESCRIPTION OF THE FIGURES

FIG. 1. Schematic illustration of a semi-custom tumor-informed assay for cancer monitoring according to embodiments of the present invention.

FIG. 2. Schematic illustration of a semi-custom tumor-uninformed assay for cancer monitoring according to embodiments of the present invention.

### DETAILED DESCRIPTION OF THE INVENTION

Methylation in the human genome occurs in the form of 5-methyl cytosine and is confined to cytosine residues that are part of the sequence CG, also denoted as CpG dinucleotides (cytosine residues that are part of other sequences are not methylated). Some CG dinucleotides in the human genome are methylated, and others are not. In addition, methylation is cell and tissue specific, such that a specific CG dinucleotide can be methylated in a certain cell and at the same time unmethylated in a different cell, or methylated in a certain tissue and at the same time unmethylated in different tissues. DNA methylation is an important regulator of gene transcription.

The methylation pattern of cancer DNA differs from that of normal DNA, wherein some loci are hypermethylated while others are hypomethylated. In some embodiments, the present invention provides methods and systems for sensitive detection of differentially methylated (e.g., hypermethylated) genomic loci associated with cancer.

### DNA Sample

DNA samples for use according to the present invention include cell-free DNA extracted from a biological fluid sample of a subject, and also DNA extracted from cells of a subject—normal or tumor cells.

The term “cell-free DNA” (abbreviated “cfDNA”) refers to DNA molecules which are freely circulating in body fluids and are not contained within intact cells. The origin of cfDNA is not fully understood but believed to be related to apoptosis, necrosis and active release from cells. cfDNA is released by both normal and tumor cells. cfDNA is highly fragmented, with an average length of approximately 150 base pairs. It is to be understood that the term “cell-free DNA” as used herein refers to DNA which is already cell-free in the body of the subject.

Biological fluid samples include plasma, serum, urine, cerebrospinal fluid, semen, stool, sputum and amniotic fluid. Each possibility represents a separate embodiment of the present invention. In particular embodiments, the cell-free DNA is from plasma. The term “plasma” refers to the liquid remaining after a whole blood sample is subjected to a separation process to remove the blood cells. Plasma samples for use according to the present invention may be samples separated from whole blood using any method of separation, including for example by centrifugation and/or filtration. Plasma samples for use according to the present invention may be collected using conventional collection containers or tubes.

DNA extracted from cells include DNA extracted from tissue/organ samples and DNA extracted from blood cells. Typically, cell lysis is required in order to extract the DNA. DNA may be obtained from tumor samples or from healthy tissues. A “tumor sample” as used herein encompasses a whole tumor resected by surgery or portions thereof. A “tumor sample” also encompasses a sample taken from a tumor by biopsy, and a sample taken from a lesion or a tissue suspected of being cancerous. Tumor samples for use according to the present invention include fresh tumor samples as well as frozen/preserved tumor samples.

A “subject” according to the present invention is typically a human subject. The subject according to the present invention is diagnosed with cancer. “Diagnosed with cancer” encompasses a subject that received a cancer diagnosis but did not yet receive treatment. The term further encompasses a subject that received a cancer diagnosis and already received a treatment. In some embodiments, the subject received a cancer diagnosis and undergone tumor resection. In some embodiments, the subject undergone tumor resection but did not receive any additional treatment (e.g., chemotherapy, radiotherapy, immunotherapy). In some embodiments, the treatment is a treatment administered in cycles, and the subject received at least one cycle. In some embodiments, the treatment is a treatment administered in cycles, and the subject completed all treatment cycles. In some embodiments, the subject is in need of monitoring for the recurrence of the cancer. In some embodiments, the subject is in need of monitoring the subject's response to a treatment.

Preferably, the DNA sample on which the methylation analysis is carried out is substantially free of single-stranded DNA (ssDNA). As used herein, “substantially free of ssDNA” or “substantially devoid of ssDNA” indicates a DNA sample in which less than 7% of the DNA is ssDNA, preferably less than 5% of the DNA is ssDNA, more preferably less than 1% of the DNA is ssDNA (namely, at least 99% of the DNA is double-stranded) (by number of molecules). In some embodiments, the DNA sample contains less than 0.1% ssDNA. In some embodiments, the DNA sample contains less than 0.01% ssDNA. In some embodiments, the DNA sample contains no ssDNA (free of ssDNA). Extraction of DNA to obtain a DNA sample substantially free of ssDNA is described, for example, in WO 2020/188561, assigned to the Applicant of the present invention.

An exemplary kit for extracting cell-free DNA which is suitable for use with the methods of the present invention is QIAamp® Circulating Nucleic Acid Kit (QIAGEN, Hilden, Germany). An exemplary kit for extracting DNA from cells is QIAamp® Blood Mini Kit.

In some embodiments, all DNA that was extracted is used according to the present invention. In some embodiments, the DNA is not quantified prior to the methylation analysis according to the present invention. In other embodiments, the DNA is quantified prior to analysis thereof. In some embodiments, the DNA is aliquoted, e.g., into a first aliquot that is subjected to a certain treatment and a second aliquot that is kept as an untreated control.

### Methylation Value

A “methylation value” as used herein is a numerical value representing the level of methylation of a particular genomic locus in a DNA sample. As methylation may be analyzed and measured using various methods, a “methylation value” according to the present invention may be expressed as a variety of numerical values. For example, a methylation value may be a methylation level expressed as a ratio or percentage of the DNA molecules that are methylated at a marker locus out of the total number of DNA molecules containing the marker locus in the sample. As a further example, a methylation value may be a methylation level expressed as a copy number of methylated DNA molecules at the marker locus (e.g., read count obtained following sequencing). As yet a further example, a methylation value may be a methylation level expressed as an intensity of a signal obtained from a marker locus, e.g., fluorescent signal obtained using a detectable fluorescent label/probe. A methylation value may be normalized with respect to a reference locus and/or a reference DNA sample. In some particular embodiments, the methylation value is a methylation ratio between a marker locus and a control locus, expressed as a ratio between signals obtained for these loci following methylation-sensitive enzymatic digestion of the DNA sample and PCR amplification, as will be described in more detail below.

The following sections describe methylation analyses according to some embodiments of the present invention, which are based on methylation-sensitive enzymatic digestion of the DNA sample followed by quantitative PCR amplification and analysis of amplification products, or methylation-sensitive enzymatic digestion of the DNA sample followed by high-throughput sequencing (next-generation sequencing). A person of skill in the art would appreciate that other methods for analyzing methylation and obtaining methylation value(s) of marker loci may be used with the present invention.

### Methylation Analysis Using Methylation-Sensitive Enzymatic Digestion and PCR Amplification

### A. DNA Digestion

According to some embodiments of the present invention, following extraction the DNA is subjected to digestion with at least one methylation-sensitive restriction endonuclease. For example, one, two or three methylation-sensitive restriction endonucleases may be used. Each number of endonucleases used in the assay represents a separate embodiment of the present invention.

In some embodiments, the entire DNA that was extracted is used in the digestion step. In some embodiments, the DNA is not quantified prior to being subjected to digestion. In other embodiments, the DNA is quantified prior to digestion thereof. In some embodiments, the DNA is aliquoted into a first aliquot that is subjected to digestion and a second aliquot that is kept as an undigested control.

A “restriction endonuclease”, used herein interchangeably with a “restriction enzyme”, refers to an enzyme that cuts DNA at or near specific recognition sequences, also known as restriction sites. Restriction sites are usually 4 to 8 nucleotide long and are typically palindromic (i.e., the DNA sequences are the same in both directions).

A “methylation-sensitive” restriction endonuclease is a restriction endonuclease that cleaves its recognition sequence only if it is unmethylated (while methylated sites remain intact). Thus, the extent of digestion of a DNA sample by a methylation-sensitive restriction endonuclease depends on the methylation level, where a higher methylation level protects from cleavage and accordingly results in less digestion.

Examples of methylation-sensitive restriction endonucleases that may be used according to the present invention include HinPlI, HhaI and AciI. Each possibility represents a separate embodiment of the present invention.

In some embodiments, a DNA sample according to the present invention is subjected to digestion with a single methylation-sensitive restriction endonuclease. In additional embodiments, a DNA sample according to the present invention is subjected to digestion with a plurality of methylation-sensitive restriction endonucleases, for example, two methylation-sensitive restriction endonucleases. In some embodiments, the plurality of methylation-sensitive restriction endonucleases comprises HinPlI. In additional embodiments, the plurality of methylation-sensitive restriction endonucleases comprises HhaI. In additional embodiments, the plurality of methylation-sensitive restriction endonucleases comprises AciI. In some particular embodiments, HinPlI and AciI are used.

In some embodiments, DNA digestion may be carried out to complete digestion. Complete digestion may be achieved following one to two hours incubation with the enzyme(s) at 37° C.

### B. Amplification of Genomic Loci

The terms “genomic locus” and “locus” as used herein are interchangeable and refer to a DNA sequence at a specific position within the genome. The specific position may be identified by the molecular location, namely, by the chromosome and the numbers of the starting and ending base pairs on the chromosome. A variant of a DNA sequence at a given genomic position is called an allele. Alleles of a locus are located at identical sites on homologous chromosomes. Genomic loci include gene sequences as well as other genetic elements (e.g., intergenic sequences).

A “marker locus” as disclosed herein refers to a genomic locus that is differentially methylated between sources of DNA, and therefore analysis of its methylation provides an indication with respect to the source of the DNA.

Marker loci disclosed herein include pan-cancer marker loci, which are genomic loci differentially methylated between normal and cancer DNA of multiple types and stages, and are therefore useful as general markers of cancer.

Marker loci disclosed herein also include cancer-specific marker loci, which are differentially methylated between different cancer types.

Available information about marker loci that can be used with the present invention include, for example:
Genome—Wide Analysis of DNA Methylation and the Gene Expression Change in Lung Cancer—ScienceDirect
Whole—genome methylation analysis of benign and malignant colorectal tumours—Beggs—2013—The Journal of Pathology—Wiley Online Library
Enduring epigenetic landmarks define the cancer microenvironment (cshlp.org)

DNA methylation markers panel can improve prediction of response to neoadjuvant chemotherapy in luminal B breast cancer|Scientific Reports (nature.com).

In some embodiments, the marker loci disclosed herein contain differentially methylated CG dinucleotides located within recognition site(s) of at least one methylation-sensitive restriction enzyme. A methylation-sensitive restriction enzyme cleaves its recognition sequence only if it is unmethylated. Thus, differences in methylation levels between DNA sources result in differences in the degree of digestion, and subsequently different amplification patterns in subsequent amplification and quantification steps. Such differences enable distinguishing between DNA from different sources, for example, between DNA samples from subjects with cancer and DNA samples from healthy subjects.

A “control locus” and “internal reference locus” are interchangeable and used herein to describe a locus, the digestion of which with the restriction enzyme applied in the digestion step is independent of the presence or absence of methylation. In some embodiments, the control locus is a locus devoid of the nucleotide sequence recognized by the at least one restriction enzyme applied in the digestion step, and the sequence of the control locus remains intact regardless of its methylation status when the DNA sample is digested. Thus, the sequence of the control locus exhibits the same digestion and amplification pattern in DNA from different sources, e.g., in normal and cancer DNA. Advantageously, the control locus is an internal locus, i.e., a locus within the analyzed DNA sample, thus eliminating the need for external/additional control sample(s).

In some embodiments, the methods of the present invention comprise amplifying at least one marker locus and at least one control locus following digestion of the DNA sample.

As used herein, “amplification” refers to an increase in the number of copies of one or more particular nucleic acid target of interest. Amplification is typically performed by polymerase chain reaction (PCR) in the presence of a PCR reaction mixture which may include a suitable buffer supplemented with the DNA template, polymerase (usually Taq Polymerase), dNTPs, primers and probes (as appropriate).

The term “polynucleotide” as used herein include polymeric forms of nucleotides of any length, either deoxyribonucleotides or ribonucleotides, or analogs thereof. The term “oligonucleotide” is also used herein to include a polymeric form of nucleotides, typically of up to 100 bases in length.

An “amplification product” collectively refers to nucleic acid molecules of a particular target sequence that are generated and accumulated in an amplification reaction. The term generally refers to nucleic acid molecules generated by PCR using a given set of amplification primers.

As used herein, a “primer” defines an oligonucleotide which is capable of annealing to (hybridizing with) a target sequence, thereby creating a double stranded region which can serve as an initiation point for DNA synthesis under suitable conditions. The terminology “primer pair” refers herein to a pair of oligonucleotides which are selected to be used together in amplifying a selected nucleic acid sequence by one of a number of types of amplification processes, preferably PCR. The primers may be designed to bind to a complementary sequence under selected conditions.

The primers may be of any suitable length, depending on the particular assay format and the particular needs. In some embodiments, the primers may include at least 15 nucleotides in length, preferably between 15-25 nucleotides in length, more preferably between 18-25 nucleotides in length. The primers may be adapted to be especially suited to a chosen nucleic acid amplification system. The oligonucleotide primers may be designed by taking into consideration the melting point of hybridization thereof with their targeted sequence.

In some embodiments, the marker and control loci may be amplified from the same DNA sample (the digested sample) using pairs of reverse and forward primers to specifically amplify each locus. In some embodiments, the primers may be designed to amplify a locus along with 5′ and 3′ flanking sequences thereof.

In some embodiments, the 5′ flanking sequences may include between 1-60 bases immediately upstream of the locus. In additional embodiments, the 5′ flanking sequences may include between 1-35 bases immediately upstream of the locus.

In some embodiments, the 3′ flanking sequences may include between 1-60 bases immediately downstream of the locus. In additional embodiments, the 3′ flanking sequences may include between 20-60 bases immediately downstream of the locus.

In some embodiments, the primers may be designed to generate amplification products of between 30-150 bps in length when the locus is intact. In some particular embodiments, the primers may be designed to generate amplification products of between 80-150 bps in length.

In some embodiments, the methods of the present invention involve simultaneous amplification of more than one target sequence (at least one marker locus and a control locus) in the same reaction mixture, a process known as multiplex amplification or co-amplification. This process requires simultaneous use of multiple primer pairs. The primers may be designed such that they can work at the same annealing temperature during amplification. In some embodiments, primers with similar melting temperature (Tm) are used in the methods disclosed herein. A Tm variation of between about 3°−5° C. is considered acceptable for primers used in a pool.

In some embodiments, all marker and control loci may be amplified in a single reaction mixture. In other embodiments, for example due to technical limitation of a particular machine, the digested DNA sample may be divided into several aliquots, each of which is supplemented with primer pairs for amplification of one or more marker loci and the control locus. Thus, even if a DNA sample is divided into several aliquots, the control locus is amplified in each aliquot, and calculation of signal ratios is performed for the control locus and a marker locus that were amplified together, i.e., from the same aliquot.

In some embodiments, amplification of the genomic loci may be carried out using real-time PCR, also known as quantitative PCR (qPCR), in which simultaneous amplification and detection of the amplification products are performed.

In some embodiments, detection of the amplification products in real-time PCR may be achieved using polynucleotide probes, typically fluorescently-labeled polynucleotide probes.

As used herein, “polynucleotide probes” or “oligonucleotide probes” are interchangeable and refer to labeled polynucleotides which are complementary to specific sub-sequences within the nucleic acid sequences of loci of interest, for example, within the sequence of a marker locus or a control locus. In some embodiments, detection is achieved by using TaqMan assays based on combined reporter and quencher molecules (Roche Molecular Systems Inc.). In such assays, the polynucleotide probes have a fluorescent moiety (fluorophore) attached to their 5′ end and a quencher attached to the 3′ end. During PCR amplification, the polynucleotide probes selectively hybridize to their target sequences on the template, and as the polymerase replicates the template it also cleaves the polynucleotide probes due to the polymerase's 5′-nuclease activity. When the polynucleotide probes are intact, the close proximity between the quencher and the fluorescent moiety normally results in a low level of background fluorescence. When the polynucleotide probes are cleaved, the quencher is decoupled from the fluorescent moiety, resulting in an increase of intensity of fluorescence. The fluorescent signal correlates with the amount of amplification products, i.e., the signal increases as the amplification products accumulate.

As used herein, “selectively hybridize to” (as well as “selective hybridization,” “specifically hybridize to,” and “specific hybridization”) refers to the binding, duplexing, or hybridizing of a nucleic acid molecule (such as a primer or a probe) preferentially to a particular complementary nucleotide sequence under stringent conditions. The term “stringent conditions” refers to conditions under which a nucleic acid molecule will hybridize preferentially to its target sequence and to a lesser extent to, or not at all to, other non-target sequences. A “stringent hybridization” in the context of nucleic acid hybridization is sequence-dependent, and differs under different conditions, as known in the art.

Polynucleotide probes may vary in length. In some embodiments, the polynucleotide probes may include between 15-30 bases. In additional embodiments, the polynucleotide probes may include between 25-30 bases. In some embodiments, the polynucleotide probes may include between 20-30 bases, for example, 20 bases, 21 bases, 22 bases, 23 bases, 24 bases, 25 bases, 26 bases, 27 bases, 28 bases, 29 bases, 30 bases. Each possibility represents a separate embodiment of the present invention.

Polynucleotide probes may be designed to bind to either strand of the template. Additional considerations include the Tm of the polynucleotide probes, which should preferably be compatible to that of the primers. Computer software may be used for designing the primers and probes.

As noted above, the methods disclosed herein may involve simultaneous amplification of more than one target sequence (at least one marker locus and a control locus) in the same reaction mixture. In order to distinguish between multiple target sequences that are amplified in parallel, polynucleotide probes labeled with distinct fluorescent colors may be used. In some embodiments, the polynucleotide probes form fluorophore/quencher pairs as known in the art, and include, for example, FAM-TAMRA, FAM-BHQ1, Yakima Yellow-BHQ1, ATTO550-BHQ2 and ROX-BHQ2. In some embodiments, the dye combinations may be compatible to the real-time PCR thermocycler of choice.

In some embodiments, fluorescence may be monitored during each PCR cycle, providing an amplification plot showing the change of fluorescent signals from the probes as a function of cycle number.

In the context of real-time PCR, the following terminology is used:

“Quantification cycle” (“Cq”) refers to the cycle number in which fluorescence increases above a threshold, set automatically by software or manually by the user. In some embodiments, the threshold may be constant for all loci and may be set in advance, prior to carrying out the amplification and detection. In other embodiments, the threshold may be defined separately for each locus after the run, based on the maximum fluorescence level detected for this locus during the amplification cycles.

“Threshold” refers to a value of fluorescence used for Cq determination. In some embodiments, the threshold value may be a value above baseline fluorescence, and/or above background noise, and within the exponential growth phase of the amplification plot.

“Baseline” refers to the initial cycles of PCR where there is little to no change in fluorescence.

Computer software may be used to analyze amplification plots and determine baseline, threshold and Cq.

Following digestion with the at least one methylation-sensitive restriction enzyme, marker loci in which CG dinucleotide(s) in the enzyme's recognition site are methylated are amplified to a high level, because the DNA molecules are protected from cleavage. The result is relatively low Cq values because detectable amplification products are shown following a relatively small number of amplification cycles. Conversely, marker loci in which CG dinucleotide(s) in the enzyme's recognition site are unmethylated are cut more extensively during the digestion step, and thus result in higher Cq values in the amplification and quantification step (i.e., show detectable amplification products following a relatively large number of amplification cycles).

In alternative embodiments, amplification and detection of amplification products may be carried out by conventional PCR using fluorescently-labeled primers followed by capillary electrophoresis of amplification products. In some embodiments, following amplification the amplification products are separated by capillary electrophoresis and fluorescent signals are quantified. In some embodiments, an electropherogram plotting the change in fluorescent signals as a function of size (bp) or time from injection may be generated, wherein each peak in the electropherogram corresponds to the amplification product of a single locus. The peak's height (provided for example using “relative fluorescent units”, rFU) may represent the intensity of the signal from the amplified locus. Computer software may be used to detect peaks and calculate the fluorescence intensities (peak heights) of a set of loci whose amplification products were run on the capillary electrophoresis machine, and subsequently the ratios between the signal intensities.

For DNA samples digested with a methylation-sensitive restriction enzyme, marker loci in which CG dinucleotide(s) in the enzyme's recognition site are methylated produce a relatively strong signal (higher peak) in the electropherogram. Conversely, marker loci in which the CG dinucleotide(s) in the enzyme's recognition site are unmethylated produce a relatively weak signal (lower peak) in the electropherogram.

In some embodiments, the fluorescent labels of the primers include any one of fluorescein, FAM, lissamine, phycoerythrin, rhodamine, Cy2, Cy3, Cy3.5, Cy5, Cy5.5, Cy7, FluorX, JOE, HEX, NED, VIC and ROX.

### C. Signal Ratio

The term “ratio” or “signal ratio” as used herein refers to the ratio between the intensities of signals obtained from co-amplification of a pair of genomic loci in the same DNA sample (in the same reaction mixture), particularly co-amplification of a marker locus and a control locus. A signal ratio between a marker locus and a control locus of the present invention, obtained following methylation-sensitive enzymatic digestion and co-amplification, reflects the methylation ratio between these marker and control loci, and represents a “methylation value” according to the present invention.

The term “signal intensity” as used herein refers to a measure reflecting the amount of locus-specific amplification products corresponding to the initial amount of intact copies of the locus. However, the signal intensity may not indicate actual amounts of amplification products/intact loci, and may not involve calculation of any absolute amounts of amplification products/intact loci. Thus, for calculating ratios of amplicon signals, no standard curve or reference DNA may be needed since it is unnecessary to calculate actual DNA concentrations or DNA methylation level per se.

In some exemplary embodiments, amplification and detection of amplification products are carried out by real-time PCR, where the signal intensity of a specific locus may be represented by the Cq calculated for this locus. The signal ratio in this case may be represented by the following calculation:

2^{(Cq of control locus-Cq of marker locus)}.

In additional exemplary embodiments, detection of amplification products is carried out by capillary electrophoresis wherein the signal intensity of a specific locus is the number of relative fluorescence units (rFUs) of its corresponding peak. The signal ratio may be calculated by dividing the heights of peaks of each marker locus by the height of the peak of a control locus.

In some embodiments, calculating a ratio between signal intensities of the amplification products of a marker locus and a control locus in a DNA sample comprises: (i) determining the signal intensity of the amplification product of the marker locus; (ii) determining the signal intensity of the amplification product of the control locus; and (iii) calculating a ratio between the two signal intensities.

In some embodiments, calculating a ratio between signal intensities of the amplification products of a marker locus and a control locus in the DNA sample comprises determining the Cq for each locus, and calculating the difference between the Cq of the control locus and the Cq of the marker locus. In some embodiments, the calculating further comprises applying the following formula: 2\hat{ }(Cq of control locus−Cq of marker locus).

In some embodiments, calculating a signal ratio may be calculating a plurality of signal ratios, between each marker locus and a control locus.

In some embodiments, computer software may be used for calculating a ratio between signal intensities of amplification products.

As noted above, a signal ratio between a marker locus and a control locus of the present invention, obtained following methylation-sensitive enzymatic digestion and co-amplification, reflects the methylation ratio between these marker and control loci, and represents a “methylation value” according to the present invention.

### Methylation Analysis Using Methylation-Sensitive Enzymatic Digestion and High-Throughput Sequencing

“High throughput sequencing” (also termed “next generation sequencing”, abbreviated “NGS”) includes sequence determination using methods that determine many (typically thousands to billions) of nucleic acid sequences in parallel. High throughput sequencing generally involves three basic steps: library preparation, sequencing and data analysis. Examples of high throughput sequencing techniques include sequencing-by-synthesis and sequencing-by-ligation (employed, for example, by lllumina Inc., Life Technologies Inc., Roche), nanopore sequencing methods and electronic detection-based methods such as Ion Torrent™ technology (Life Technologies Inc.).

According to some embodiments of the present invention, following extraction the DNA is subjected to digestion with at least one methylation-sensitive restriction endonuclease as described herein, and subsequently a sequencing library is prepared.

In some embodiments, preparing a sequencing library comprises introducing adapter oligonucleotides, also termed “sequencing adapters”, to DNA fragments, and enriching DNA fragments corresponding to marker loci of interest and optionally one or more control loci.

Enrichment of genomic regions of interest may be carried out, for example, using locus-specific PCR, or using capture agents in a solution-phase or a solid-phase hybridization-based process.

The sequencing adapters are oligonucleotides at the 5′ and 3′ ends of each DNA fragment in a sequencing library. Sequencing adapters typically include platform-specific sequences for fragment recognition by a particular sequencer: for example, sequences that enable library fragments to bind to the flow cells of Illumina platforms. Each sequencing instrument typically employs a specific set of sequences for this purpose. The sequencing adapters may include sample indices, which are sequences that enable multiple samples to be sequenced together (i.e., multiplexed) on the same instrument flow cell or chip. Each sample index, typically 6-10 bases, is specific to a given sample library and is used for de-multiplexing during data analysis to assign individual sequence reads to the correct sample. Sequencing adapters may contain single or dual sample indexes depending on the number of libraries combined and the level of accuracy desired.

Sequencing adapters may be introduced into analyzed DNA fragments by ligation or via PCR. In some embodiments, a 2-step PCR is used, in order to enrich genomic regions of interest and introduce sequencing adapters to the enriched fragments. The first PCR is carried out using primers that contain locus-specific sequences and overhang sequences that introduce a first portion of the sequencing adapters, and the second PCR is carried out using primers that introduce a second portion of the sequencing adapters and optionally sample indices.

A sequencing library is subjected to sequencing to obtain multiple sequence reads. The sequence reads are analyzed using a computer software to determine a read count (copy number) for each locus of interest. The read count of a marker locus reflects the number of methylated copies of this locus that were present in the tested DNA sample (the methylated copies remain intact when the sample is digested with methylation-sensitive restriction endonucleases). Relative copy number may be calculated for a marker locus, for example, with respect to a control locus, as follows:

Relative copy number=Read count of marker locus/Read count of control locus

A read count of a marker locus or alternatively a relative copy number of a marker locus represent “methylation values” according to the present invention.

In some embodiments, analysis of methylation values according to the present invention comprises:
(a) subjecting a DNA sample to digestion with at least one methylation-sensitive restriction endonuclease, thereby obtaining restriction endonuclease-treated DNA;
(b) generating a sequencing library from the restriction endonuclease-treated DNA, the sequencing library comprising DNA fragments corresponding to at least one marker locus as disclosed herein (preferably a plurality of marker loci as disclosed herein) and at least one control locus;
(c) subjecting the sequencing library to high-throughput sequencing and determining a copy number for each of the at least one marker locus and the control locus; and
(d) comparing a ratio between the copy numbers of each of said at least one marker locus and the control locus to at least one reference ratio, to assess the presence of cancer in a subject and/or indicate a tissue source of the cancer.

In some embodiments, generating a sequencing library comprises enriching DNA fragments corresponding to the at least one marker locus and the at least one control locus.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1—Design of a Panel of Genomic Loci for Assaying a Lung Cancer Patient

A set of 90 human genomic loci is identified, comprising genomic loci that are hypermethylated across a broad range of cancer types including lung cancer, and genomic loci that are hypermethylated specifically in lung cancer. These DNA methylation marker loci are more methylated in DNA of cancer cells compared to normal non-cancer cells.

Advantageously, the marker loci are suitable for methylation analysis using methylation-sensitive enzymatic digestion of DNA samples followed by real-time PCR amplification and analysis of the amplification products. More particularly, each marker locus contains at least one restriction site of at least one methylation-sensitive restriction endonuclease, preferably a plurality of restriction sites of a plurality of methylation-sensitive restriction endonucleases. The restriction sites within the marker loci are differentially methylated between cancer and non-cancer DNA. Methylation-sensitive restriction endonucleases cleave their restriction site only if it is unmethylated. Thus, the degree of digestion of each locus by the at least one methylation-sensitive restriction endonuclease depends on its level of methylation in a tested DNA sample, where increased methylation results in less digestion.

PCR primers and probes for amplification and detection of each marker locus in the panel are designed and validated. In addition, primers and probe for amplification and detection of a control locus are designed and validated, the control locus does not contain a nucleotide sequence that is recognized by the at least one methylation-sensitive restriction endonuclease.

### Example 2—Semi-Custom Tumor-Informed Assay for Lung Cancer Monitoring

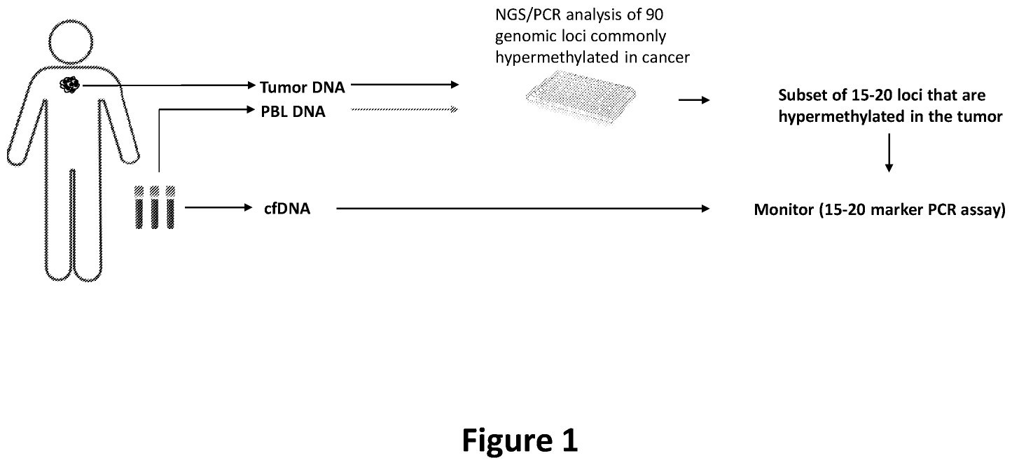

A schematic illustration of a semi-custom tumor-informed assay according to some embodiments of the present invention is shown in FIG. 1.

First, the methylation level of the defined set of 90 genomic loci described in Example 1 is analyzed in tumor DNA and in normal DNA (from peripheral blood leukocytes, PBLs) of a subject diagnosed with lung cancer. Next, a subset of 15-20 genomic loci with the highest methylation in the tumor DNA compared to the normal DNA is selected from the set of 90 genomic loci. This subset of genomic loci represents the most informative genomic loci for the subject's tumor out of the complete set. The tumor is monitored non-invasively by testing the selected subset of genomic loci in cell-free DNA from plasma sample(s) of the subject.

Testing the panel of genomic loci in DNA from tumor samples, PBLs and plasma samples:

Tumor samples from a plurality of lung cancer patients are obtained and DNA is extracted from each sample. In addition, DNA is extracted from peripheral blood leukocytes of the same patients, serving as a corresponding normal (non-cancer) DNA. The tumor DNA samples and their corresponding normal DNA samples are analyzed to obtain a methylation value for each marker locus in the set of 90 marker loci described above. For each tumor sample, a subset of the most informative marker loci is defined, by selecting 15-20 marker loci out of the 90 marker loci that have the highest methylation values in the tumor DNA compared to the corresponding normal DNA.

In parallel, plasma samples of the same patients collected pre-operatively and prior to administration of any treatment are obtained. DNA is extracted from the plasma samples, and each plasma DNA sample is analyzed to obtain a methylation value for each marker locus in the set of 90 marker loci described above. For each plasma DNA sample, a subset of 15-20 marker loci with the highest methylation values is selected out of the 90 marker loci. The subset selected for each plasma DNA sample is compared to the subset selected for the corresponding tumor sample (namely, for the tumor sample of the same patient), to identify a correlation between the subsets. A correlation indicates that a subset of marker loci selected based on tumor DNA of a subject is suitable for non-invasive monitoring of the tumor in a cell-free DNA sample of the subject.

### Testing a Selected Subset of Marker Loci

The pre-operative plasma samples are analyzed using only the subset of 15-20 most informative marker loci, to test whether the presence of the tumor can be identified using this subset. A methylation value for each locus in the subset of 15-20 marker loci is determined. A tumor is “detected” when a combined score of the subset is above a predefined threshold. The correct identification of the presence of the tumor demonstrates that a subset of marker loci selected based on tumor DNA is suitable for non-invasive monitoring of the tumor in a cell-free DNA sample.

The methylation analysis of DNA from tumor, PBLs and plasma samples is carried out using the same method, as follows:

DNA is extracted and subsequently digested with at least one methylation-sensitive restriction endonuclease recognizing a sequence within each marker locus that is differentially methylated between cancer and non-cancer DNA. Real-time PCR is carried out on the digested DNA to amplify marker loci and a control locus that does not contain a recognition sequence of the at least one methylation-sensitive restriction endonuclease used in the digestion step. The complete set of 90 genomic loci commonly hypermethylated in cancer is amplified, or only a selected subset of 15-20 genomic loci which are the most informative for a particular tumor, as required.

Following amplification, data on the level of fluorescent signals from the probes as a function of cycle number are analyzed to calculate the quantification cycle (Cq) for each locus, and a ΔCq between the Cq of the control locus and the Cq of a marker locus is calculated for each marker locus and used in the following formula:

2^{(Cq of control locus-Cq of marker locus)}

It is to be understood that the calculation is performed for a marker locus and a control locus that are co-amplified in the same aliquot.

The numerical value obtained for a given marker locus with respect to the control locus represents a ratio between the signal intensities of the amplification products of this marker locus and the control locus, and reflects the methylation ratio between this marker locus and the control locus in the DNA sample. The signal ratio represents a methylation value of the marker locus.

### Example 3—Semi-Custom Tumor-Uninformed Assay for Lung Cancer Monitoring

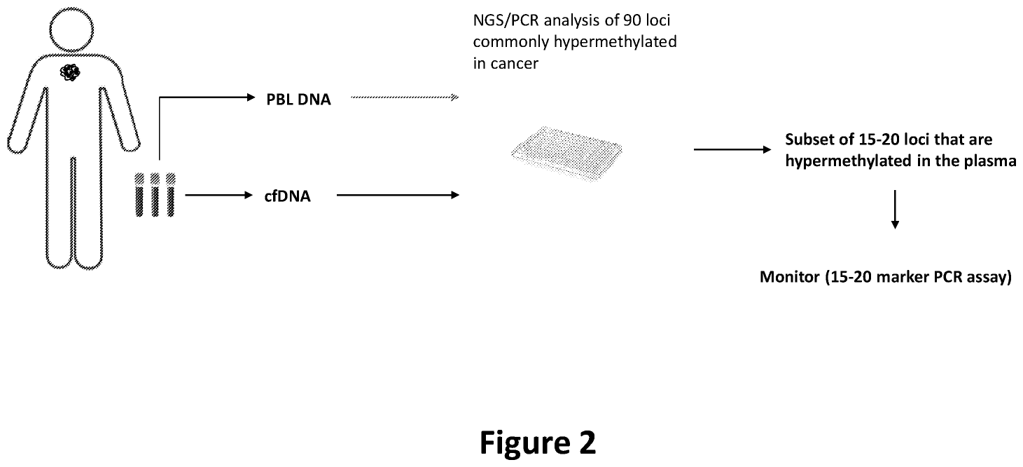

A schematic illustration of a semi-custom tumor-uninformed assay according to some embodiments of the present invention is shown in FIG. 2. The tumor-uninformed configuration does not require analyzing a tumor sample in order to select a subset of informative markers. The assay is based on analysis of cell-free DNA and DNA from peripheral blood leukocytes (PBLs) of a subject diagnosed with cancer.

First, the methylation of the defined set of 90 genomic loci described above is analyzed in cell-free DNA from a plasma sample and in DNA from PBLs of a subject diagnosed with cancer. Next, a subset of 15-20 genomic loci with the highest methylation in the cell-free DNA compared to the PBL DNA is selected from the set of 90 genomic loci. This subset of genomic loci represents the most informative genomic loci for the subject's tumor out of the complete set. The tumor is monitored non-invasively by testing the selected subset of genomic loci in cell-free DNA sample(s) of the subject (e.g., plasma DNA).

### Testing Cell-Free Plasma DNA and PBL DNA:

Plasma samples are obtained from a plurality of lung cancer patients pre-operatively and prior to administration of any treatment, and DNA is extracted from each sample. In addition, DNA is extracted from peripheral blood leukocytes (PBLs) of the same patients. The cell-free DNA samples from the plasma and their corresponding DNA samples from the PBLs are analyzed to obtain a methylation value for each marker locus in the set of 90 marker loci described above. For each cell-free DNA sample, a subset of the most informative marker loci is defined, by selecting 15-20 marker loci out of the 90 marker loci that have the highest methylation values in the cell-free DNA compared to the corresponding PBL DNA.

The methylation analysis of DNA from PBLs and plasma samples is carried out using methylation-sensitive enzymatic digestion of the DNA followed by real-time PCR amplification and analysis of amplification products, as described above.

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. A method for establishing a personalized panel of cancer-associated DNA methylation markers, the method comprising:
(a) determining in a tumor DNA sample of a subject diagnosed with cancer a methylation value for each marker locus in a pre-defined set of marker loci comprising pan-cancer marker loci, cancer-specific marker loci which are specific to the cancer of the subject, or a combination thereof;
(b) comparing the methylation values determined in step (a) to corresponding methylation values of the marker loci in normal non-cancer DNA; and
(c) selecting a subset of marker loci out of the set of marker loci that show a significant difference in methylation values in the tumor DNA compared to the corresponding normal non-cancer DNA, thereby establishing a personalized panel of cancer-associated DNA methylation markers.

2. The method of claim 1, wherein the subject is diagnosed with a solid tumor cancer, and wherein the methylation values of the marker loci in normal non-cancer DNA are methylation values determined in DNA extracted from peripheral blood leukocytes of the subject.

3. The method of claim 1, wherein the subject is diagnosed with a hematological cancer, and wherein the methylation values of the marker loci in normal non-cancer DNA are methylation values determined in DNA extracted from urine or saliva samples of the subject.

4. The method of claim 1, wherein the subject is diagnosed with a hematological cancer, and wherein the methylation values of the marker loci in normal non-cancer DNA are reference methylation values determined based on a plurality of DNA samples of healthy subjects without a hematological cancer.

5. The method of claim 1, wherein methylation values are determined using methylation-sensitive enzymatic digestion of DNA followed by high-throughput sequencing.

6. The method of claim 1, wherein methylation values are determined using methylation-sensitive enzymatic digestion of DNA followed by quantitative PCR and analysis of amplification products.

7. A method for guiding therapy of a subject diagnosed with cancer, the method comprising:
(i) establishing for the subject a personalized panel of cancer-associated DNA methylation markers according to claim 1; and
(ii) analyzing the methylation values of the selected subset of marker loci in one or more cell-free DNA samples of the subject to guide therapy of the subject, wherein methylation values of the selected subset of marker loci in the cell-free DNA sample are indicative of tumor burden in the subject.

8. The method of claim 7, wherein the cell-free DNA sample is cell-free DNA extracted from a plasma sample.

9. The method of claim 8, wherein guiding therapy of the subject comprises providing an indication of recurrence of the cancer, and wherein analyzing the methylation values of the selected subset of marker loci in one or more cell-free DNA samples of the subject comprises:
(i) determining baseline methylation values of the selected subset of marker loci in a first cell-free DNA sample of the subject collected at a first time point after administration of a treatment;
(ii) determining methylation values of the selected subset of marker loci in a second cell-free DNA sample of the subject collected at a second later time point after administration of the treatment; and
(iii) comparing the methylation values determined in the second cell-free DNA sample to the baseline methylation values in order to provide an indication whether the cancer recurred, wherein an increase in methylation values is indicative of cancer recurrence;
(iv) optionally repeating steps (ii)-(iii) for cell-free DNA samples collected at additional time points after administration of the treatment, and further comparing the methylation values determined at a late time point to methylation values determined at earlier time points, wherein an increase in methylation values is indicative of cancer recurrence.

10. The method of claim 9, further comprising:
administering to the subject active cancer surveillance and follow-up testing when the indication for cancer recurrence is positive, for definitive diagnosis of cancer recurrence and optionally monitoring the progression of the cancer, wherein the cancer surveillance and follow-up testing comprises one or more of blood tests, urine tests, cytology, imaging, endoscopy and biopsy.

11. The method of claim 10, further comprising administering treatment when the definitive diagnosis shows cancer recurrence, wherein the treatment comprises one or more of surgical resection, chemotherapy, radiation therapy, immunotherapy, and targeted therapy

12. The method of claim 8, wherein guiding therapy of the subject comprises assessing response to treatment, and wherein analyzing the methylation values of the selected subset of marker loci in one or more cell-free DNA samples of the subject comprises:
(i) determining methylation values of the selected subset of marker loci in a first cell-free DNA sample of the subject collected before administration of a treatment;
(ii) determining methylation values of the selected subset of marker loci in a second cell-free DNA sample of the subject collected after administration of the treatment; and
(iii) comparing the methylation values determined after treatment to the methylation values determined before treatment in order to assess response to the treatment, wherein a decrease in methylation values is indicative of a positive response to the treatment; and
(iv) optionally repeating steps (ii)-(iii) for cell-free DNA samples collected at additional time points after administration of the treatment, and further comparing the methylation values determined at a late time point to methylation values determined at earlier time points in order to monitor response to the treatment, wherein an increase in methylation values is indicative of a positive response to the treatment.

13. The method of claim 12, wherein the treatment comprises surgery and the second cell-free DNA sample is a sample taken after the surgery.

14. The method of claim 12, wherein the treatment is a treatment administered in cycles and the second cell-free DNA sample is one or more samples taken after one or more cycles.

15. The method of claim 12, wherein the treatment is a treatment administered in cycles and the second cell-free DNA sample is a sample taken after the cycles are completed.

16. The method of claim 8, wherein guiding therapy of the subject comprises predicting response to a treatment, and wherein analyzing methylation values of the selected subset of marker loci in one or more cell-free DNA samples of the subject comprises:
(i) determining methylation values of the selected subset of marker loci in a cell-free DNA sample of the subject collected before administration of the treatment; and
(ii) comparing the methylation values determined in the cell-free DNA sample of the subject to reference methylation values determined in a plurality of reference subjects that received the treatment, to classify the likelihood of the subject to positively respond to the treatment.

17. A method of administering a cancer treatment to a subject diagnosed with cancer, the method comprising:
classifying the likelihood of the subject to respond positively to the treatment according to the method of claim 16; and
administering the treatment to the subject when the subject is classified as likely to respond positively to the treatment.

18. The method of claim 8, wherein guiding therapy of the subject comprises classifying minimal residual disease after administration of a treatment, and wherein analyzing methylation values of the selected subset of marker loci in one or more cell-free DNA samples of the subject comprises:
(i) determining methylation values of the selected subset of marker loci in a first cell-free DNA sample of the subject collected before administration of the treatment;
(ii) determining methylation values of the selected subset of marker loci in a second cell-free DNA sample of the subject collected after administration of the treatment; and
(iii) comparing the methylation values determined after treatment to the methylation values determined before treatment and to methylation values in a normal non-cancer DNA, to classify minimal residual disease.

19. The method of claim 18, wherein the treatment comprises surgery and the second cell-free DNA sample is a sample taken after the surgery.

20. The method of claim 18, wherein the treatment is a treatment administered in cycles and the second cell-free DNA sample is a sample taken after the cycles are completed.

21-29. (canceled)

30. A method for personalized analysis of cancer-related methylation changes in DNA samples of a subject diagnosed with cancer, the method comprising:
(A) providing a set of reagents for quantifying methylation of a pre-defined set of marker loci in DNA samples, the pre-defined set of marker loci comprises pan-cancer marker loci, cancer-specific marker loci which are specific to the cancer of the subject, or a combination thereof;
(B) determining in a tumor DNA sample of the subject a methylation value for each marker locus in the pre-defined set, and identifying a subset of marker loci from the set that show a significant difference in methylation values in the tumor DNA compared to corresponding methylation values of the marker loci in normal non-cancer DNA;
(C) selecting from the set of reagents provided in step (A) those reagents that are suitable for quantifying methylation of the subset of marker loci identified in step (B); and
(D) using the selected reagents for analyzing the methylation values of the subset of marker loci in one or more cell-free DNA samples of the subject, thereby performing a personalized analysis of cancer-related methylation changes in DNA samples of the subject.

31. (canceled)

32. A method for establishing a personalized panel of cancer-associated DNA methylation markers, the method comprising:
(a) determining in cell-free DNA extracted from a plasma sample of a subject diagnosed with cancer a methylation value for each marker locus in a pre-defined set of marker loci comprising pan-cancer marker loci, cancer-specific marker loci which are specific to the cancer of the subject, or a combination thereof;
(b) comparing the methylation values determined in step (a) to corresponding methylation values of the marker loci in normal non-cancer DNA; and
(c) selecting a subset of marker loci out of the set of marker loci that show a significant difference in methylation values in the cell-free DNA compared to the corresponding normal non-cancer DNA, thereby establishing a personalized panel of cancer-associated DNA methylation markers.

33-36. (canceled)
